(19) United States
(12) Patent Application Publication
(10) Pub. No.: US 2005/0084540 A1
(43) Pub. Date: Apr. 21, 2005

(54) Taste masking antibiotic composition

(76) Inventors: Indranil Nandi, Plainsboro, NJ (US); Mintong Guo, Plainsboro, NJ (US); Chad Michael Gassert, Hoboken, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/688,551

(22) Filed: Oct. 17, 2003

(51) Int. Cl. A61K 9/16; A61K 9/50

(52) U.S. Cl. 424/490

(57) ABSTRACT

A taste masking composition comprising micropellets containing an antibiotic wherein said micropellets have an inner coating comprising at least one cellulose polymer which is not an enteric coating polymer and an outer coating comprising an enteric coating polymer, wherein said micropellets have a particle size of about 100 μm to about 650 μm. The micropellets are characterized by (i) a lack of bitter taste; (ii) fast dissolution; and (iii) increased bioavailability of antibiotic as compared to antibiotic containing pellets having a drug-polymer matrix.

### FIELD OF THE INVENTION

The present invention provides a taste masking composition comprising micropellets containing an antibiotic wherein said micropellets have an inner coating comprising at least one cellulose polymer which is not an enteric coating polymer and an outer coating comprising an enteric coating polymer, wherein said micropellets have a particle size of about 100 μm to about 650 μm.

### BACKGROUND OF THE INVENTION

Antibiotics such as clarithromycin and erythromycin has been used in the treatment of common pediatric infections of the middle ear and upper respiratory tract, as well as certain forms of pneumonia that affects the elderly. However, such antibiotics are extremely bitter, and even when dissolved in trace quantities in a liquid dosage form are often perceived to be unpalatable. Administration of such antibiotics to children and the elderly poses a challenge as these patients experience difficulty in swallowing solid oral dosage forms. For these patients, antibiotics are typically provided in liquid forms, such as solutions, emulsions, and suspensions, which usually permit perceptible exposure of the antibiotic to the taste bud.

There is a need to mask the taste of such antibiotics in order to ensure patient compliance during therapy. Conventional taste masking techniques, such as the use of sweeteners, amino acids, and flavoring agents often are unsuccessful in masking the taste of highly bitter drugs and, consequently, other techniques need to be exploited for effectively masking the taste of these antibiotics.

One such technique involves the use of cation exchange resins, such as polysulfonic acid and polycarboxylic acid polymers, to adsorb amine drugs for taste masking and sustained release. However, this technique has limited applicability and is not capable of masking the taste of highly bitter drugs.

Coating of bitter drugs is another method which has been reported for taste masking. This technique alone may prove effective for moderately bitter drugs or in products where the coated particles are formulated as aqueous preparations before administration or are formulated in a non-aqueous medium. This technique has its limitations as coating of fine particles is usually technology intensive and coated granules are readily ruptured by chewing and compression.

Lipid-based microencapsulation is another technique used to taste mask the drugs. This technique requires highly sophisticated hot-melt granulation for producing fine particles, and may have adverse effects on heat sensitive molecules or restrict drug release adversely. U.S. Pat. No. 4,865,851 describes cefuroxime axetil in particulate form coated with an integral coating of lipid or a mixture of lipids.

U.S. Pat. No. 4,808,411 describes a taste-masked composition in the form of granules which contain clarithromycin and a carbomer acrylic acid polymer. The clarithromycin and carbomer are believed to be held together by both the ionic interactions between the amine group of clarithromycin and the carbonyl group of the carbomer and by the gel properties of the carbomer. This complex is further taste masked by coating.

U.S. Pat. No. 5,286,489 describes a porous drug-polymer matrix formed by admixing one or more bitter tasting active ingredient and a methyl methacrylic ester copolymer in at least a 1:1 by weight ratio of active ingredient to copolymer, effective to mask the taste of the drug. None of the examples described in U.S. Pat. No. 5,286,489 describe the effect of such polymers on the release of the drug from the matrix. While such a drug-polymer matrix may result in good taste-masking, the matrix may also retard the rate of drug release from the matrix to an extent which would be unacceptable for a conventional immediate-release formulation.

U.S. Patent No. 6,565,877 describes a taste-masked composition containing a bitter tasting drug, such as clarithromycin, and a combination of two enteric polymers comprising a methacrylic acid copolymer and a phthalate polymer, wherein the ratio of methacrylic acid copolymer to phthalate polymer is between 1:9 or 9:1.

International Application WO 03/082248 describes a pharmaceutical composition containing erythromycin A or a derivative thereof, such as clarithromycin, and alginic agid.

International Application WO 03/082241 describes a pharmaceutical composition containing micronized clarithromycin. The clarithromycin has a particle size less than 35 microns.

### SUMMARY OF THE INVENTION

The invention provides a taste masking composition comprising micropellets containing an antibiotic wherein said micropellets have an inner coating comprising at least one cellulose polymer which is not an enteric coating polymer and an outer coating comprising an enteric coating polymer, wherein said micropellets have a particle size of about 100 μm to about 650 μm, and said antibiotic preferably has a particle size of about 0.1 μm to about 100 μm.

According to another aspect, the invention provides a method for preparing a taste masking composition comprising micropellets containing an antibiotic, said method comprising high-shear granulation in the presence of an impeller set at least at 50 rpm, said micropellets having an inner coating comprising at least one cellulose polymer which is not an enteric coating polymer and an outer coating comprising an enteric coating polymer, wherein said micropellets have a particle size of about 100 μm to about 650 μm. Preferably, the high-shear granulation is additionally conducted in the presence of a chopper set at least at 1000 rpm.

According to another aspect, the invention provides a method for preparing a taste masking composition comprising micropellets containing an antibiotic, said method comprising
(a) mixing at least one antibiotic, and optionally one or more excipients, to form a premix;
(b) adding a solvent, and optionally one or more excipients, to the premix formed in Step (a) and granulating in the presence of an impeller, to form a wet granulation;
(c) drying the wet granulation, and optionally milling and screening the dried granules to form micropellets; and
(d) coating the micropellets with at least one cellulose polymer; and
(e) coating the micropellets from Step (d) with at least one enteric coating polymer to form coated micropellets .

The antibiotic containing micropellets of the invention are characterized by (i) a lack of bitter taste; (ii) fast dissolution; and (iii) increased bioavailability of the antibiotic as compared to antibiotic containing pellets having a drug-polymer matrix.

### DESCRIPTION OF THE INVENTION

The invention provides a taste masking antibiotic composition comprising antibiotic containing micropellets having an inner coating comprising at least one cellulose polymer which is not an enteric coating polymer and an outer coating comprising an enteric coating polymer. As used herein, “micropellets” refers to granules having a particle size of about 100 μm to about 650 μm, preferably 200 μm to about 450 μm. The antibiotic preferably has a particle size of about 0.1 μm to about 100 μm, more preferably 5 μm to about 40 μm.

Preferred antibiotics include the following: erythromycin; clarithromycin; fluoroquinolones, such as ciprofloxacin and norfloxacin; cephalosporins, such as cefuroxime and ceftriaxone; and tetracyclic antibiotics, for example, chloramphenicol, chlorpromazine, etc. A combination of antibiotics may also be used. Preferably, the antibiotic is clarithromycin.

The antibiotic is present in an amount of from about 1 wt. % to about 80 wt. %, based on the total weight of the micropellet. Preferably, the antibiotic is present in an amount of from about 5 wt. % to about 50 wt. %, more preferably, about 20 wt. % to about 35 wt. %, based on the total weight of the micropellet.

Preferred cellulose polymers include the following: hydroxypropylmethyl cellulose, hydroxypropyl cellulose, methyl cellulose, ethyl cellulose, carboxymethylethyl cellulose, sodium carboxymethyl cellulose, and ethylcarboxyethyl cellulose. A combination of cellulose polymers may also be used. More preferably, the cellulose polymer is hydroxypropylmethyl cellulose or hydroxypropyl cellulose. Most preferably, the cellulose polymer is hydroxypropylmethyl cellulose.

Preferred enteric coatings include the following: cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene copolymer; polyvinylalcohols; polyoxyethyleneglycols; polyethylene glycol; sodium alginate; galactomannone; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. EUDRAGIT®-L and -S series, such as L100-55, L30D55, L100, S100, L12,5, and S12,5, available from Rohm; polyvinyl acetate; fats; oils; waxes; fatty alcohols; shellac; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane. A combination of enteric coatings may also be used.

More preferably, the enteric coating is selected from a copolymer of methacrylic acid and methyl methacrylate, and a copolymer of methacrylic acid and ethyl acrylate. Most preferably, the enteric coating is poly(methacrylic acid, ethyl acrylate)1:1 (EUDRAGIT®-L30D 55 and EUDRAGIT®-L100-55).

It is within the scope of the invention for the taste masking antibiotic composition to include one or more pharmaceutically acceptable excipients. Examples of such excipients are binders, diluents, anti-caking agents, amino acids, fillers, solubilizers, disintegrants, lubricants, emulsifiers, flavorants, solvents, stabilizers, anti-oxidants, anti-adherents, preservatives, electrolytes, and glidants. A combination of excipients may also be used. Such excipients are known to those skilled in the art, and thus, only a limited number will be specifically referenced.

Preferred binders include, but are not limited to, starches, e.g., potato starch, wheat starch, corn starch; gums, such as gum tragacanth, acacia gum and gelatin; and polyvinyl pyrrolidone, e.g., Povidone. Polyvinyl pyrrolidone is a particularly preferred binder.

Preferred fillers include, but are not limited to, microcrystalline cellulose, starch, pregelatinized starch, modified starch, dibasic calcium phosphate dihydrate, calcium sulfate trihydrate, calcium sulfate dihydrate, calcium carbonate, dextrose, sucrose, lactose, mannitol, and sorbitol. Lactose is a particularly preferred filler.

Examples of disintegrants include:
(i) natural starches, such as maize starch, potato starch and the like; directly compressible starches, e.g., Starch 1500; modified starches, e.g., carboxymethyl starches and sodium starch glycolate, available as Primojel®, Explotab®, Explosol®; and starch derivatives, such as amylose;
(ii) cross-linked polyvinylpyrrolidones, e.g., crospovidones, such as Polyplasdone® XL and Kollidon® CL;
(iii) sodium alginate;
(iv) methacrylic acid-divinylbenzene co-polymer salts, e.g., Amberlite® IRP-88; and
(v) cross-linked sodium carboxymethylcellulose, available as, e.g., Ac-Di-Sol®, Primellose®, Pharmacel® XL, Explocel® and Nymcel® ZSX.
   Additional disintegrants also include hydroxypropyl cellulose; hydroxypropylmethyl cellulose; croscarmellose sodium; sodium starch glycolate; polacrillin potassium; polyacrylates, such as Carbopol®; magnesium aluminium silicate; and bentonite. A preferred disintegrant is Starch 1500.

The taste masking composition comprising micropellets containing an antibiotic are prepared by high-shear granulation in the presence of an impeller set at least at 50 rpm. Preferably, the impeller is set at about 300 rpm. Preferably, the high-shear granulation is additionally conducted in the presence of a chopper which preferably is set at least at 1000 rpm, more preferably the chopper is set at about 2400 rpm. Following granulation, the micropellets are dried and screened. The microparticles obtained after screening are coated with an inner coating comprising at least one cellulose polymer which is not an enteric coating polymer and an outer coating comprising an enteric coating polymer.

In one embodiment of the invention, the taste masking composition comprising micropellets containing an antibiotic is prepared by a method comprising:
(a) mixing at least one antibiotic, and optionally one or more excipients, to form a premix;
(b) adding a solvent, and optionally one or more excipients, to the premix formed in Step (a) and granulating in the presence of an impeller set at least at 50 rpm, and a chopper, to form a wet granulation;
(c) drying the wet granulation, and optionally milling and screening the dried granules to form micropellets; and
(d) coating the micropellets with at least one cellulose polymer; and
(e) coating the micropellets from Step (d) with at least one enteric coating polymer to form coated micropellets.

Drying techniques useful for drying granules include spray-drying, fluid bed drying, flash drying, ring drying, micron drying, tray drying, vacuum drying, radio-frequency drying and microwave drying. A preferred drying technique is fluid bed.

Milling is a process of reducing larger size granules to smaller size granules in order to achieve proper flow. Types of mills which may be used in the invention include, but are not limited to, fluid energy mill, ball mill or rod mill, hammer mill, cutting mill and oscillating granulator. More specifically, suitable mills include, Quadro, Fryma, Glatt Quick Sieve, Fluidaire, Fitzpatrick (Fitz mill), BTS mill and Tornado. A preferred mill is a Fitz mill.

The pharmaceutical compositions of the invention may be in the form of an oral suspension, capsule, caplet, powder, or tablet. In a preferred embodiment, the pharmaceutical compositions are in the form of an oral suspension.

The following non-limiting examples illustrate further aspects of the invention.

### EXAMPLE 1

### Preparation of a Clarithromycin Composition

| Ingredient | Amount |
|---|---|
| Clarithromycin | 250.0 g |
| Lactose Monohydrate | 90.0 g |
| Starch 1500 | 95.0 g |
| Croscarmellose Na | 80.0 g |
| Polyvinylpyrrolidone K-90 | 6.0 g |
| Purified Water | q.s. |

The clarithromycin, lactose, starch and croscarmellose Na were mixed in a 2.5 L high-shear VG5 Glatt granulator for 5 minutes with an impeller set at 350 rpm and chopper set at 2000 rpm. Separately, the polyvinylpyrrolidone was mixed with water at room temperature until dissolved. The polyvinylpyrrolidone solution was added over a period of three minutes to the mixture containing clarithromycin and mixed in the granulator at 250 mL/min at the above settings. Mixing in the granulator was continued for an additional three minutes at the above settings to form wet granules. The wet granules were discharged and placed on a tray which was placed in an oven at 55° C. for 4 hours to form dried granules. The dried granules were screened through U.S. Standard Sieve No. 30, 40, 50, and 80 mesh screens. The granules collected on the 30 mesh screen were milled using a Quadro Co-mill equipped with a screen #62 to form micropellets. The micropellets were subjected to the screening procedure as described above and the particle size distribution was summarized in Table I. The yield of micropellets remaining on Sieves Nos. 40 to 80 was determined to be 83.5%, based on the total amount of ingredients.

TABLE I

| Sieve No. | Amount (g) |
|---|---|
| 30 | 18.6 |
| 40 | 207.1 |
| 50 | 136.7 |
| 80 | 49.7 |
| Pan | 59.2 |

### EXAMPLE 2

### Preparation of Clarithromycin Composition

| Ingredient | Amount | |
|---|---|---|
| Clarithromycin | 250.0 | g |
| Lactose, regular | 90.0 | g |
| Starch 1500 | 95.0 | g |
| Ac-Di-Sol | 80.0 | g |
| Polyvinylpyrrolidone K-90 | 6.0 | g |
| Water | 415 | mL |

The clarithromycin, lactose, starch and Ac-Di-Sol were mixed in a 2.5 L high-shear VG5 Glatt granulator for 5 minutes with a impeller set at 300 rpm and chopper set at 2400 rpm. Separately, the polyvinylpyrrolidone was mixed with water at room temperature until dissolved. The polyvinylpyrrolidone solution was added over a period of three minutes to the mixture containing clarithromycin and mixed in the granulator at 250 mL/min at the above settings. Mixing in the granulator was continued for an additional three minutes at the above settings for form wet granules. The wet granules were discharged and placed on a tray which was placed in an oven at 55° C. for 4 hours to form dried granules. The dried granules were screened through U.S. Standard Sieve No. 30, 40, 50, and 80 mesh screens. The granules collected on the 30 mesh screen were milled using a Fitzpatrick Mill equipped with a screen #65 to form micropellets. The micropellets were subjected to the screening procedure as described above and the particle size distribution was summarized in Table II. The yield of micropellets remaining on Sieves Nos. 40 to 80 was determined to be 81.15%, based on the total amount of ingredients.

TABLE II

| Sieve No. | Amount (g) |
|---|---|
| 20 | 4.0 |
| 30 | 57.1 |
| 40 | 120.7 |
| 50 | 179.8 |
| 60 | 29.2 |
| 80 | 36.0 |
| Pan | 45.8 |

### EXAMPLE 3

### Preparation of Clarithromycin Composition

| Ingredient | Amount | |
|---|---|---|
| Clarithromycin | 250.0 | g |
| Lactose, regular | 75.0 | g |
| Starch 1500 | 80.0 | g |
| Ac-Di-Sol | 80.0 | g |
| Polaxomer 188 | 34.0 | g |
| Polyvinylpyrrolidone K-90 | 6.0 | g |
| Water | 400 | mL |

The clarithromycin, lactose, starch and Ac-Di-Sol were mixed in a 2.5 L high-shear VG5 Glatt granulator for 5 minutes with an impeller set at 400 rpm and no chopper blade. Separately, the polyvinylpyrrolidone and Poloxamer 188 were mixed with water at room temperature until dissolved. The polyvinylpyrrolidone and polaxomer 188 solution was added over a period of fifteen minutes to the mixture containing clarithromycin and mixed in the granulator at 62 mL/min at the above settings. Mixing in the granulator was continued for an additional three minutes at the above settings to form wet granules. The wet granules were discharged and placed on a tray which was placed in an oven at 55° C. for 4 hours to form dried granules. The dried granules were screened through U.S. Standard Sieve No. 30, 40, 50, and 80 mesh screens. The granules collected on the 30 mesh screen were milled using a Fitzpatrick Mill equipped with a screen #65 to form micropellets. The micropellets were subjected to the screening procedure as described above and the particle size distribution was summarized in Table III. The yield of micropellets remaining on Sieves Nos. 40 to 80 was determined to be 58.0%, based on the total amount of ingredients.

TABLE III

| Sieve No. | Amount (g) |
|---|---|
| 20 | 36.0 |
| 30 | 82.5 |
| 40 | 100.7 |
| 50 | 130.6 |
| 60 | 29.2 |
| 80 | 40.0 |
| Pan | 57.4 |

### EXAMPLE 4

### Preparation of Inner Coating (Cellulose Polymer)

| Ingredient | Amount | |
|---|---|---|
| Hydroxypropylmethyl cellulose | 40 | g |
| Water | 226 | mL |
| Simethicone | 1 | g |

Hydroxypropylmethyl cellulose, water and simethicone were mixed.

### EXAMPLE 5

### Preparation of Outer Coating (Enteric Coating Polymer)

| Ingredient | Amount | |
|---|---|---|
| Eugragit L30 D55 | 419.25 | g |
| Polysorbate 80 | 1.50 | g |
| Glyceryl Monostearate | 3.75 | g |
| Triethyl Citrate | 18.75 | g |
| Water | 306.38 | mL |

Polysorbate 80, 1.5 g, was dissolved in 250 mL water with heating at 70° C. Glyceryl monostearate, 3.75 g, was added to the polysorbate solution at 70° C. and mixed. The mixture was allowed to cool with agitation. Eugragit L 30 D55, 419.25 g, which is in the form of a 30% aqueous dispersion was screened through a U.S. Sieve No. 40 mesh screen and the particles collected on the No. 40 mesh screen were collected. Triethyl Citrate, 18.75 g, was mixed with 56.38 mL of water to form a solution which was combined with the Eugragit dispersion, and added to the mixture containing polysorbate 80 and glyceryl monostearate, with agitation.

### EXAMPLE 6

### Preparation of Coated Micropellets

The micropellets prepared in Example 1 were first coated with a cellulose polymer coating composition as prepared in Example 4 using a Wuster Column in a Glatt Fluid Bed Granulator. The coated micropellets were further coated with an enteric coating composition as prepared in Example 5 using a Wuster Column in a Glatt Fluid Bed Granulator. The coated micropellets were subjected to the screening procedure as described above and the particle size distribution is summarized in Table IV.

TABLE IV

| Sieve No. | Amount (g) |
|---|---|
| 30 | 15.8 |
| 40 | 80.6 |
| 50 | 73.7 |
| 60 | 20.6 |
| 80 | 20.7 |
| Pan | 12.1 |

While the invention has been described with particular reference to certain embodiments thereof, it will be understood that changes and modifications may be made by those of ordinary skill within the scope and spirit of the following claims.

1. A taste masking composition comprising micropellets containing an antibiotic wherein said micropellets have an inner coating comprising at least one cellulose polymer which is not an enteric coating polymer and an outer coating comprising an enteric coating polymer, wherein said micropellets have a particle size of about 100 μm to about 650 μm.

2. The composition according to claim 1 wherein the antibiotic has a particle size of about 0.1 μm to about 100 μm.

3. The composition according to claim 1, wherein the cellulose polymer is selected from the group consisting of hydroxypropylmethyl cellulose, hydroxypropyl cellulose, methyl cellulose, ethyl cellulose, carboxymethylethyl cellulose, sodium carboxymethyl cellulose, ethylcarboxyethyl cellulose, and combinations thereof.

4. The composition according to claim 3, wherein the cellulose polymer is selected from the group consisting of hydroxypropylmethyl cellulose and hydroxypropyl cellulose.

5. The composition according to claim 4, wherein the cellulose polymer is hydroxypropylmethyl cellulose.

6. The composition according to claim 1, wherein the enteric coating is selected from the group consisting of cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene copolymer; polyvinylalcohols; polyoxyethyleneglycols; polyethylene glycol; sodium alginate; galactomannone; carboxypolymethylene; sodium carboxymethyl starch; copolymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, and octadecyl acrylate; polyvinyl acetate; fats; oils; waxes; fatty alcohols; shellac; gluten; ethylacrylate-maleic acid anhydride copolymer; maleic acid anhydride-vinyl methyl ether copolymer; styrol-mateic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane, and combinations thereof.

7. The composition according to claim 6, wherein the enteric coating is selected from the group consisting of a copolymer of methacrylic acid and methyl methacrylate, and a copolymer of methacrylic acid and ethyl acrylate.

8. The composition according to claim 7, wherein the enteric coating is a poly(methacrylic acid, ethyl acrylate)1:1.

9. The composition according to claim 1, wherein the amount of antibiotic is from about 1 wt. % to about 80 wt. %, based on the total weight of the micropellet.

10. The composition according to claim 9, wherein the amount of antibiotic is from about 5 wt. % to about 50 wt. %, based on the total weight of the micropellet.

11. The composition according to claim 10, wherein the amount of antibiotic is from about 20 wt. % to about 35 wt. %, based on the total weight of the micropellet.

12. The composition according to claim 1 in the form of an oral suspension, capsule, caplet, powder, or tablet.

13. An oral suspension comprising the composition according to claim 1.

14. A method for preparing a taste masking composition comprising micropellets containing an antibiotic, said method comprising high-shear granulation in the presence of an impeller set at least at 50 rpm, said micropellets having an inner coating comprising at least one cellulose polymer which is not an enteric coating polymer and an outer coating comprising an enteric coating polymer, wherein said micropellets have a particle size of about 100 μm to about 650 μm.

15. A method for preparing a taste masking composition comprising micropellets containing an antibiotic, said method comprising
(a) mixing at least one antibiotic, and optionally one or more excipients, to form a premix;
(b) adding a solvent, and optionally one or more excipients, to the premix formed in Step (a) and granulating in the presence of an impeller set at least at 50 rpm, to form a wet granulation;
(c) drying the wet granulation, and optionally milling and screening the dried granules to form micropellets; and
(d) coating the micropellets with at least one cellulose polymer; and
(e) coating the micropellets from Step (d) with at least one enteric coating polymer to form coated micropellets .

16. The method according to claim 14, wherein the high-shear granulation is additionally conducted in the presence of a chopper.

17. The method according to claim 16, wherein the chopper is set at least at 1000 rpm.

18. The method according to claim 15 wherein the solvent is water.

19. The method according to claim 15 wherein the antibiotic is clarithromycin.

20. The method according to claim 15 wherein the excipient is selected from the group consisting of a binder, diluent, anti-caking agent, amino acid, filler, solubilizer, disintegrant, lubricant, emulsifier, flavorant, solvent, stabilizer, anti-oxidant, anti-adherent, preservative, electrolyte, glidant, and combinations thereof.
